(19) United States
(12) Patent Application Publication
(10) Pub. No.: US 2019/0388350 A1
(43) Pub. Date: Dec. 26, 2019

(54) FORMULATION

(71) Applicant: SEKISUI CHEMICAL CO., LTD., Osaka-City (JP)

(72) Inventors: Saori Tone, Osaka (JP); Kazushi Itou, Osaka (JP); Takayuki Akamine, Osaka (JP); Daichi Kawamura, Osaka (JP); Yoshiko Abe, Tokyo (JP)

(21) Appl. No.: 16/016,507

(22) Filed: Jun. 22, 2018

(51) Int. Cl. A61K 9/16; A61K 31/445; A61K 31/53; A61K 31/27; A61K 31/381

(52) U.S. Cl. CPC A61K 9/1611; A61K 9/1694; A61K 31/445; A61K 31/381; A61K 9/1617; A61K 31/53; A61K 31/27; A61K 9/1623

(57) ABSTRACT

Problem to be Solved

There is provided a formulation including a particle containing an active ingredient and a surfactant, wherein morphological stability is excellent.

Solution

A particle containing an active ingredient, a surfactant and a pH adjuster.

### TECHNICAL FIELD

The present invention relates to a formulation, and particularly relates to a formulation for use in an external medicine, a cosmetic product, and the like.

### BACKGROUND ART

There is used an external medicine (systemically acting external medicine) which exerts a systemic action by migration to systemic circulation of a drug absorbed through the skin, or an external medicine (topically acting external medicine) which topically exerts a medical effect with a drug absorbed through the skin. There is also used a cosmetic product (percutaneous absorption cosmetic product) for the purpose of percutaneous absorption of an active ingredient. A main technical problem of such a formulation includes those about storage stability and/or skin permeability of an active ingredient, and there is proposed a formulation containing a particle mainly constituted from an active ingredient and a surfactant (Patent Literatures 1 and 2). Such a particle can be usually obtained by homogenizing a water (W) phase containing an active ingredient and an oil (0) phase containing a surfactant to provide a W/O emulsion, and lyophilizing the emulsion, or the like.

### CITATION LIST

### Patent Literatures

[Patent Literature 1] Japanese Patent Laid-Open No. 2009-084293

[Patent Literature 2] International Publication No. WO 2006/025583

### SUMMARY OF INVENTION

### Technical Problem

Meanwhile, the present inventors have found that a formulation containing a particle mainly constituted from an active ingredient and a surfactant may be observed to cause a morphological change of the particle over time.

An object of the present invention is to provide a formulation including a particle containing an active ingredient and a surfactant, wherein morphological stability is excellent.

### Solution to Problem

The present inventors have made intensive studies in order to solve the above problem, and thus have found that the above problem can be solved by adjusting the pH of a water phase containing an active ingredient in production of a particle mainly constituted from an active ingredient and a surfactant. The present invention has been completed through further trial and error based on such a finding, and includes the following aspects.

### Aspect 1.

A particle containing an active ingredient, a surfactant and a pH adjuster.

### Aspect 2.

The particle according to aspect 1, wherein

the particle contains a first fraction and a second fraction, and

the first fraction contains the active ingredient and the pH adjuster.

### Aspect 3.

The particle according to aspect 1 or 2, wherein the pH adjuster is at least one pH adjuster selected from the group consisting of an inorganic acid and an organic acid, and a salt thereof.

### Aspect 4.

The particle according to any one of aspects 1 to 3, wherein the pH adjuster is at least one pH adjuster selected from the group consisting of hydrochloric acid and a hydrochloride.

### Aspect 5.

The particle according to any one of aspects 1 to 4, including 0.01 to 2% by weight of the pH adjuster based on the entire particle.

### Aspect 6.

The particle according to any one of aspects 1 to 5, wherein the active ingredient is hydrophilic.

### Aspect 7.

A formulation including the particle according to any one of aspects 1 to 6.

### Aspect 8.

A method for producing a particle containing an active ingredient, a surfactant and a pH adjuster, the method including:

(1) a step of preparing a water (W) phase containing an active ingredient and a pH adjuster;

(2) a step of homogenizing the water (W) phase and an oil (O) phase containing a surfactant to provide a W/O emulsion; and

(3) a step of drying the W/O emulsion.

### Advantageous Effect of Invention

According to the present invention, there can be provided a formulation including a particle mainly constituted from an active ingredient and a surfactant, wherein morphological stability of the particle is excellent.

### BRIEF DESCRIPTION OF DRAWINGS

FIG. 1 is an image observed by an optical microscope of a formulation obtained in Example 1.

FIG. 2 is an image observed by an optical microscope of a formulation obtained in Comparative Example 1.

FIG. 3 is an image observed by an optical microscope of a formulation obtained in Comparative Example 2.

### DESCRIPTION OF EMBODIMENTS

### 1. Configuration of Formulation

The formulation of the present invention contains at least the following particle.

### 1.1 Particle

The particle contains an active ingredient and a surfactant. More specifically, the particle contains at least two fractions of a first fraction containing an active ingredient and a second fraction containing a surfactant.

In the particle, a part or the entire surface of the first fraction containing an active ingredient is covered with the surfactant of the second fraction. The particle can have such a configuration, thereby allowing the formulation of the present invention to slowly release the active ingredient of the first fraction.

Herein, the first fraction and the second fraction may be connected to each other (preferably by an intermolecular force) to form an aggregate. The entire surface of the first fraction may be covered with the second fraction, or the entire surface of the first fraction may not be covered with the second fraction.

Examples of aspects of the particle include a core-shell structure where the first fraction corresponds to a core portion and the second fraction corresponds to a shell portion including the core portion. In the core-shell structure, the entire surface of the core portion is not necessarily covered with the shell portion.

1.1.1 First fraction

The first fraction includes at least an active ingredient and a pH adjuster.

The active ingredient is not particularly limited as long as it is an ingredient having physiological activity. Preferably, the active ingredient is an ingredient compounded for the purpose of exertion of physiological activity. In such a preferable aspect, the active ingredient does not encompass an ingredient which, while having physiological activity, is not compounded for the purpose of exertion of the physiological activity, in terms of the amount compounded, a compounding method, and the like. Examples of the active ingredient include an ingredient which is compounded as an active ingredient in a pharmaceutical product, a cosmetic product, or the like. The active ingredient may be an organic substance because most of active ingredients of pharmaceutical products and cosmetic products are organic substances.

The active ingredient is not particularly limited, and can be widely selected.

The active ingredient is preferably hydrophilic.

When the active ingredient is hydrophilic, an active ingredient having the following properties can be typically used, but is not particularly limited:

the molecular weight is 10000 or less and

the octanol-water partition coefficient is −8 to 6.

In the foregoing, the molecular weight is preferably 5000 or less, more preferably 2000 or less. The lower limit of the molecular weight is not particularly limited, and is usually 50 or more.

In the foregoing, the octanol-water partition coefficient is preferably −6 to 5, more preferably −5 to 4.

In the present invention, the octanol-water partition coefficient is calculated according to the following expression, by adding the active ingredient into a flask in which octanol and an aqueous buffer of pH 7 are placed, thereafter shaking it, and determining the active ingredient concentrations of the respective phases.

Octanol-water partition coefficient=Log₁₀

### (Concentration in Octanol Phase/Concentration in Water Phase)

When the active ingredient is a drug, a drug demanded for systemic action or topical action is suitably used, but is not particularly limited.

Specific examples of the active ingredient compounded to a pharmaceutical product include, but are not particularly limited, an antidementia drug, an antiepileptic drug, an antidepressant, an antiparkinsonian drug, an antiallergic drug, an anticancer drug, an antidiabetic drug, an antihypertensive drug, an erectile dysfunction drug, a dermatosis drug, a local anesthetic, and pharmaceutically acceptable salts thereof. More specific examples include donepezil, vardenafil, rivastigmine, duloxetine, galantamine, nitroglycerin, lidocaine, fentanyl, male hormones, female hormones, nicotine, clomipramine, diphenhydramine, nalfurafine, metoprolol, fesoterodine, vardenafil, nalfurafine, tandospirone, beraprost sodium, taltirelin, lurasidone, nefazodone, rifaximin, benidipine, doxazosin, nicardipine, formoterol, lomerizine, amlodipine, octreotide, teriparatide, bucladesine and cromoglicic acid, and pharmaceutically acceptable salts thereof. Such pharmaceutically acceptable salts are not particularly limited, and examples thereof include hydrochlorides such as donepezil hydrochloride, vardenafil hydrochloride and duloxetine hydrochloride, tartrates such a rivastigmine tartrate, hydrobromides such as galantamine hydrobromide, fumarates such as fesoterodine fumarate, and citrates such as tandospirone citrate.

The active ingredient, when applied to a cosmetic product, is not particularly limited as long as it is required to penetrate into the skin, and examples include vitamin ingredients such as vitamin C and vitamin E, moisturizing ingredients such as hyaluronic acid, ceramide and collagen, whitening ingredients such as tranexamic acid and arbutin, hair growth ingredients such as minoxidil, beauty ingredients such as FGF (fibroblast growth factor) and EGF (epidermal growth factor), and salts and derivatives thereof.

The amount of the active ingredient included in the particle can be, for example, 0.1 to 50% by weight based on the entire particle, depending on the type of the active ingredient.

The first fraction may contain, if necessary, two or more active ingredients. In this case, at least one active ingredient can be used as a compounding agent.

In the present invention, the pH adjuster refers to a compound which is added to an object to thereby change the pH of the object, and is not particularly limited. Preferably, the pH adjuster means a compound (pH reducing agent) which is added to an object to thereby reduce the pH of the object.

The pH adjuster is not particularly limited. For example, the pH adjuster may be at least one pH adjuster selected from the group consisting of an inorganic acid or an organic acid and salts thereof.

Specific examples of the inorganic acid include, but are not particularly limited, hydrochloric acid, sulfuric acid and phosphoric acid.

Specific examples of the organic acid include, but are not particularly limited, citric acid, adipic acid, succinic acid, tartaric acid, lactic acid, fumaric acid, malic acid, benzoic acid, acetic acid and gluconic acid.

Specific examples of the pH adjuster include, but are not particularly limited, a hydrochloride and a citrate.

The particle of the present invention preferably includes the pH adjuster so that the relative ratio between the pH adjuster and the active ingredient is as follows. That is, the particle of the present invention includes the active ingredient and the pH adjuster at a relative ratio between such ingredients so as to be lower in pH than that of an aqueous solution where the concentration of the active ingredient is 0.5% by weight. The particle of the present invention includes the pH adjuster at such a relative ratio, and thus is excellent in morphological stability.

In the foregoing, the degree of reduction in pH can be adjusted depending on the type of the active ingredient, and the reduction in pH (value) is generally preferably 1 or more, more preferably 2 or more, further preferably 3 or more, still more preferably 4 or more, but is not particularly limited thereto.

The particle of the present invention preferably includes 0.01 to 2% by weight of the pH adjuster based on the entire particle in terms of morphological stability of the particle of the present invention.

In the present invention, the pH adjuster in the particle of the present invention can be detected by a method including the following steps (1) to (5).

(1) preparing a buffer (4 mL) obtained by further adjusting the pH of a liquid containing 5×10⁻⁴ M of NaHzPO₄, 2×10⁻⁴ M of Na₂PO₄, 5×10⁻⁴ M of NaCl and 10 ppm of gentamicin sulfate (produced by Wako Pure Chemical Industries, Ltd., G1658) in distilled water, to 7.2 by NaOH.

(2) preparing a 7.5% S/O formulation according to a method described below by use of the particle of the present invention and liquid paraffin as a dispersion liquid.

(3) adding the 7.5% S/O formulation (0.8 mL) obtained in (2) above to the buffer obtained in (1).

(4) stirring the solution obtained in (3) overnight to thereby elute the agent and the pH adjuster in the S/O formulation into the buffer.

(5) detecting the pH adjuster included in the solution obtained in (4) above by liquid chromatography and the like.

The first fraction may further contain, in addition to the active ingredient and the pH adjuster, at least one other ingredient.

Examples of such other ingredient include, but are not particularly limited, a stabilizer, a percutaneous absorption enhancer, a skin irritation suppressant and an antiseptic.

The stabilizer has the action of stabilization of the particle structure, and therefore can prevent the unintentional early disintegration of the particle structure, thereby allowing the slow-release effect of the active ingredient to be retained.

Specific examples of the stabilizer include, but are not particularly limited, polysaccharide, protein and a hydrophilic polymer material. The stabilizer may contain one or more thereof. The content of the stabilizer in the first fraction can be appropriately set depending on the type thereof, and, for example, the stabilizer can also be compounded so that the weight ratio between the active ingredient and the stabilizer is 100:1 to 1:10.

Specific examples of the percutaneous absorption enhancer include, but are not particularly limited, a higher alcohol, N-acyl sarcosine and a salt thereof, a higher monocarboxylic acid, a higher monocarboxylic acid ester, an aromatic monoterpene fatty acid ester, a divalent carboxylic acid having 2 to 10 carbon atoms and a salt thereof, a polyoxyethylene alkyl ether phosphoric acid ester and a salt thereof, lactic acid, lactic acid ester, and citric acid. The percutaneous absorption enhancer may contain one or more thereof. The content of the percutaneous absorption enhancer in the core portion can be appropriately set depending on the type thereof, and, for example, the percutaneous absorption enhancer can be compounded so that the weight ratio between the active ingredient and the percutaneous absorption enhancer is 100:1 to 1:50.

Specific examples of the skin irritation suppressant include, but are not particularly limited, hydroquinone glycoside, pantethine, tranexamic acid, lecithin, titanium oxide, aluminum hydroxide, sodium nitrite, sodium hydrogen nitrite, soybean lecithin, methionine, glycyrrhetinic acid, BHT, BHA, vitamin E and a derivative thereof, vitamin C and a derivative thereof, benzotriazole, propyl gallate, and mercaptobenzimidazole. The skin irritation suppressant may contain one or more thereof. The content rate of the skin irritation suppressant in the first fraction can be appropriately set depending on the type thereof, and, for example, the skin irritation suppressant can also be compounded so that the content rate is 0.1% to 50%.

Specific examples of the antiseptic include, but are not particularly limited, methyl paraoxybenzoate, propyl paraoxybenzoate, phenoxy ethanol and thymol. The content rate of the antiseptic in the first fraction can be appropriately set depending on the type thereof, and, for example, the antiseptic can also be compounded so that the content rate is 0.01% to 10%. The antiseptic may contain one or more thereof.

### 1.1.2 Second Fraction

The second fraction includes at least a surfactant.

A plurality of surfactants may be used in combination.

As the surfactant, a surfactant where the weighted average value of the HLB (Hydrophile Lypophile Balance) value is 10 or less, preferably 5 or less, more preferably 3 or less can be used.

The surfactant preferably has a melting point of 50° C. or less, more preferably 40° C. or less in terms of absorbability of the formulation.

As the surfactant, a surfactant where the weighted average value of the HLB value is 10 or less and the melting point is 50° C. or less can be preferably used, a surfactant where the weighted average value of the HLB value is 5 or less and the melting point is 50° C. or less can be more preferably used, and a surfactant where the weighted average value of the HLB value is 5 or less and the melting point is 40° C. or less can be further preferably used.

In the present invention, the HLB value serves as an index in order to find whether an emulsifier is hydrophilic or lipophilic, and is a value of 0 to 20. It is indicated that the lower the HLB value is, the stronger the lipophilicity is. In the present invention, the HLB value is calculated from the following Griffin equation.

HLB value=20×((Molecular weight of hydrophilic moiety)/(Total molecular weight))

The weighted average value of the HLB value is calculated as follows.

For example, when there are surfactant raw materials having HLB values A, B and C, respectively, and the weights of the respective materials loaded in particle synthesis are designated as x, y and z, respectively, the calculation expression of the weighted average value is as follows:

(xA+yB+zC)÷(x+y+z)

The melting point of the surfactant in the present invention is determined from an endothermic peak in differential scanning calorimeter (DSC) measurement.

The surfactant is not particularly limited and can be appropriately selected depending on the intended use. For example, the surfactant can be widely selected from those which can be used for external medicines and/or cosmetic products.

The surfactant may be any of a nonionic surfactant, an anionic surfactant, a cationic surfactant and an amphoteric surfactant.

Examples of the nonionic surfactant include, but are not particularly limited, a fatty acid ester, a fatty alcohol ethoxylate, a polyoxyethylene alkyl phenyl ether, an alkyl glycoside and a fatty acid alkanolamide.

The fatty acid ester is preferably, but is not particularly limited, a sugar fatty acid ester. In particular, a sucrose fatty acid ester is preferable. Specific examples include esters of fatty acids such as erucic acid, oleic acid, lauric acid, stearic acid, and behenic acid, with sucrose.

Examples of such other fatty acid ester include, but are not particularly limited, ester of at least one of glycerol, polyglycerol, polyoxyethylene glycerol, sorbitan and polyoxyethylene sorbitol with fatty acid. In particular, a polyglycerol fatty acid ester is preferable.

Examples of the anionic surfactant include an alkyl sulfuric acid ester salt, a polyoxyethylene alkyl ether sulfuric acid ester salt, an alkylbenzenesulfonic acid salt, a fatty acid salt and a phosphoric acid ester salt.

Examples of the cationic surfactant include, an alkyl trimethylammonium salt, a dialkyl dimethyl ammonium salt, an alkyl dimethylbenzylammonium salt, and amine salts.

Examples of the amphoteric surfactant include an alkylamino fatty acid salt, alkyl betaine and an alkylamine oxide.

The surfactant may have an alkyl chain, but is not particularly limited thereto. The length of the alkyl chain is not particularly limited, and can be widely selected from 8 to 30 and is particularly preferably 10 to 24.

When only a surfactant having an alkyl chain is used or when a surfactant having an alkyl chain is used in combination with other surfactant, the formulation of the present invention is excellent in absorbability at a weight ratio between the total alkyl chain included in the active ingredient and that included in the surfactant, of 1:1 to 1:70. The weight ratio is preferably 1:2 to 1:50 from such a viewpoint.

The second fraction may further contain, in addition to the surfactant, at least one other ingredient. Examples of such other ingredient include, but are not particularly limited, an irritation suppressant, an analgesic, an absorption enhancer, a stabilizer and an antiseptic.

Specific examples of the irritation suppressant include, but are not particularly limited, hydroquinone glycoside, pantethine, tranexamic acid, lecithin, titanium oxide, aluminum hydroxide, sodium nitrite, sodium hydrogen nitrite, soybean lecithin, methionine, glycyrrhetinic acid, BHT, BHA, vitamin E and a derivative thereof, vitamin C and a derivative thereof, benzotriazole, propyl gallate, and mercaptobenzimidazole. The irritation suppressant may contain one or more thereof. The content rate of the irritation suppressant in the second fraction can be appropriately set depending on the type thereof, and, for example, the irritation suppressant can also be compounded so that the content rate is 0.1% to 50%.

Specific examples of the analgesic include, but are not particularly limited, local anesthetics such as procaine, tetracaine, lidocaine, dibucaine and prilocaine, and salts thereof. The analgesic may contain one or more thereof. The content rate of the analgesic in the second fraction can be appropriately set depending on the type thereof, and, for example, the analgesic can also be compounded so that the content rate is 0.1% to 30%.

Specific examples of the absorption enhancer include, but are not particularly limited, a higher alcohol, N-acyl sarcosine and a salt thereof, a higher monocarboxylic acid, a higher monocarboxylic acid ester, an aromatic monoterpene fatty acid ester, a divalent carboxylic acid having 2 to 10 carbon atoms and a salt thereof, a polyoxyethylene alkyl ether phosphoric acid ester and a salt thereof, lactic acid, lactic acid ester, and citric acid. The absorption enhancer may contain one or more thereof. The content rate of the absorption enhancer in the shell portion can be appropriately set depending on the type thereof, and, for example, the absorption enhancer can also be compounded so that the content rate is 0.1% to 30%.

The stabilizer has the action of stabilization of the particle. The stabilizer can prevent the unintentional early disintegration of the particle structure, thereby allowing the slow-release effect of a drug.

Specific examples of the stabilizer include, but are not particularly limited, a fatty acid and a salt thereof, parahydroxybenzoic acid esters such as methylparaben and propylparaben, alcohols such as chlorobutanol, benzyl alcohol and phenylethyl alcohol, thimerosal, acetic anhydride, sorbic acid, sodium hydrogen sulfite, L-ascorbic acid, sodium ascorbate, butylhydroxyanisole, butylhydroxytoluene, propyl gallate, tocopherol acetate, dl-α-tocopherol, proteins, and polysaccharides. The stabilizer may contain one or more thereof. The content of the stabilizer in the second fraction can be appropriately set depending on the type thereof, and, for example, the stabilizer can also be compounded so that the weight ratio between the surfactant and the stabilizer is 1:0.01 to 1:50.

Specific examples of the antiseptic include, but are not particularly limited, methyl paraoxybenzoate, propyl paraoxybenzoate, phenoxy ethanol and thymol. The antiseptic may contain one or more thereof. The content rate of the antiseptic in the second fraction can be appropriately set depending on the type thereof, and, for example, the antiseptic can also be compounded so that the content rate is 0.01% to 10%.

### 1.2 Base Phase

The formulation of the present invention may further contain a phase containing a base (base phase). The base phase may contain the particle. The particle may be here dispersed in the base phase.

The base can be appropriately selected from those bases suitable for dispersion of the particle, depending on the intended use and the like, and is not particularly limited.

A plurality of such base may be used in combination.

Examples of the base include, but are not particularly limited, a vegetable oil, an animal oil, a neutral lipid, a synthetic oil, a sterol derivative, waxes, hydrocarbons, monoalcohol carboxylic acid esters, oxyacid esters, polyhydric alcohol fatty acid esters, silicones, higher (polyhydric) alcohols, higher fatty acids and fluorine-based oils. These can be appropriately combined and used.

Examples of the vegetable oil include, but are not particularly limited, soybean oil, sesame oil, olive oil, coconut oil, balm oil, rice oil, cottonseed oil, sunflower oil, rice-bran oil, cacao butter, corn oil, safflower oil and rapeseed oil.

Examples of the animal oil include, but are not particularly limited, mink oil, turtle oil, fish oil, beef tallow, horse fat, pig fat and shark squalane.

Examples of the neutral lipid include, but are not particularly limited, triolein, trilinolein, trimyristin, tristearin and triarachidonin.

Examples of the synthetic oil include, but are not particularly limited, phospholipid and azone.

Examples of the sterol derivative include, but are not particularly limited, dihydrocholesterol, lanosterol, dihydrolanosterol, phytosterol, cholic acid and cholesteryl linoleate.

Examples of the waxes include, candelilla wax, carnauba wax, rice wax, Japan wax, beeswax, montan wax, ozokerite, ceresin, paraffin wax, microcrystalline wax, petrolatum, Fischer-Tropsch wax, polyethylene wax and an ethylene-propylene copolymer.

Examples of the hydrocarbons include, liquid paraffin (mineral oil), heavy liquid isoparaffin, light liquid isoparaffin, an α-olefin oligomer, polyisobutene, hydrogenated polyisobutene, polybutene, squalane, olive-derived squalane, squalene, vaseline and hard paraffin.

Examples of the monoalcohol carboxylic acid esters include, octyldodecyl myristate, hexyldecyl myristate, octyldodecyl isostearate, cetyl palmitate, octyldodecyl palmitate, cetyl octanoate, hexyldecyl octanoate, isotridecyl isononanoate, isononyl isononanoate, octyl isononanoate, isotridecyl isononanoate, isodecyl neopentanoate, isotridecyl neopentanoate, isostearyl neopentanoate, octyldodecyl neodecanoate, oleyl oleate, octyldodecyl oleate, octyldodecyl ricinoleate, lanolin fatty acid octyldodecyl, hexyldecyl dimethyloctanoate, octyldodecyl erucate, hydrogenated castor oil isostearate, ethyl oleate, avocado oil fatty acid ethyl, isopropyl myristate, isopropyl palmitate, octyl palmitate, isopropyl isostearate, lanolin fatty acid isopropyl, diethyl sebacate, diisopropyl sebacate, dioctyl sebacate, diisopropyl adipate, dibutyl octyl sebacate, diisobutyl adipate, dioctyl succinate and triethyl citrate.

Examples of the oxyacid esters include cetyl lactate, diisostearyl malate and hydrogenated castor oil monoisostearate.

Examples of the polyhydric alcohol fatty acid esters include glyceryl trioctanoate, glyceryl trioleate, glyceryl triisostearate, glyceryl diisostearate, glyceryl tri(caprylate/caprate), glyceryl tri(caprylate/caprate/myristate/stearate), hydrogenated rosin triglyceride (hydrogenated ester gum), rosin triglyceride (ester gum), glyceryl behenate/eicosadioate, trimethylolpropane trioctanoate, trimethylolpropane triisostearate, neopentylglycol dioctanoate, neopentylglycol dicaprate, 2-butyl-2-ethyl-1,3-propanediol dioctanoate, propylene glycol dioleate, pentaerythrityl tetraoctanoate, pentaerythrityl hydrogenated rosinate, ditrimethylolpropane triethyl hexanoate, ditrimethylolpropane (isostearate/sebacate), pentaerythrityl triethyl hexanoate, dipentaerythrityl (hydroxystearate/stearate/rosinate), diglyceryl diisostearate, polyglyceryl tetraisostearate, polyglyceryl-10 nonaisostearate, polyglyceryl-8 deca(erucate/isostearate/ricinoleate), (hexyldecanoic acid/sebacic acid) diglyceryl oligo ester, glycol distearate (ethylene glycol distearate), 3-methyl-1,5-pentanediol dineopentanoate and 2,4-diethyl-1,5-pentanediol dineopentanoate.

Examples of the silicones include, dimethicone (dimethylpolysiloxane), highly polymerized dimethicone (highly polymerized dimethylpolysiloxane), cyclomethicone (cyclic dimethylsiloxane, decamethylcyclopentasiloxane), phenyl trimethicone, diphenyl dimethicone, phenyl dimethicone, stearoxypropyl dimethylamine, an (aminoethylaminopropyl methicone/dimethicone) copolymer, dimethiconol, a dimethiconol crosspolymer, a silicone resin, a silicone rubber, an amino-modified silicone such as aminopropyl dimethicone or amodimethicone, a cation-modified silicone, a polyether-modified silicone such as dimethicone copolyol, a polyglycerol-modified silicone, a sugar-modified silicone, a carboxylic acid-modified silicone, a phosphoric acid-modified silicone, a sulfuric acid-modified silicone, alkyl-modified silicone, a fatty acid-modified silicone, an alkyl ether-modified silicone, an amino acid-modified silicone, a peptide-modified silicone, a fluorine-modified silicone, a cation-modified or polyether-modified silicone, an amino-modified or polyether-modified silicone, an alkyl-modified or polyether-modified silicone, and a polysiloxane-oxyalkylene copolymer.

Examples of the higher (polyhydric) alcohols include cetanol, myristyl alcohol, oleyl alcohol, lauryl alcohol, cetostearyl alcohol, stearyl alcohol, arachyl alcohol, behenyl alcohol, jojoba alcohol, chimyl alcohol, selachyl alcohol, batyl alcohol, hexyldecanol, isostearyl alcohol, 2-octyldodecanol and dimer diol.

Examples of the higher fatty acids include lauric acid, myristic acid, palmitic acid, stearic acid, isostearic acid, behenic acid, undecylenic acid, 12-hydroxystearic acid, palmitoleic acid, oleic acid, linoleic acid, linolenic acid, erucic acid, docosahexaenoic acid, eicosapentaenoic acid, isohexadecanoic acid, anteisoheneicosanoic acid, long-chain branched fatty acid, dimer acid and hydrogenated dimer acid.

Examples of the fluorine-based oils include perfluorodecane, perfluorooctane and perfluoropolyether.

Examples of other bases include, but are not particularly limited, bases for use in an ointment, a cream, an aerosol, a tape, a patch, a poultice, a gel or a microneedle.

As the base, not only the base exemplified above, but also a base containing any other ingredient may be appropriately used. A base further containing an additive (gelling agent) having a gelation action may be adopted. Such an additive is not particularly limited, and, for example, hydrocarbons such as resin and silicone, amino acid, cyclic peptide, epoxy, rosin, melamine, pectin such as polysaccharide and a surfactant, alginic acid, carrageenan, locust bean gum, guar gum, xanthan gum, a dextrin fatty acid ester, an inulin fatty acid ester, and a glyceryl fatty acid ester can be used. Examples of the resin include, but are not particularly limited, polyethylene, polypropylene, polyester, polystyrene and polyurethane. A base containing such a resin is not particularly limited, and, for example, a base containing 0.1 to 50%, preferably 1 to 30% of such an additive, can be used. Such a base is not particularly limited, and, for example, hydrocarbon type bases such as liquid paraffin, cyclohexane, n-octane, toluene and xylene, and ester type bases such as isopropyl myristate, isopropyl palmitate and cetyl ethyihexanoate are preferably used. Specific examples of such a base are not particularly limited, and, for example, a hydrocarbon gel ointment base such as Plastibase (Plastibase) (registered trademark) (Bristol Myers Squibb) including 95% of liquid paraffin, and 5% of a polyethylene resin as a gelling agent can be used.

### 1.3 Other Additive Ingredient(s)

The formulation of the present invention may further contain, in addition to the particle and the base phase, other additive ingredient(s) depending on the dosage form, the intended use, and the like.

Examples of the additive ingredient include, but are not particularly limited, an excipient, a colorant, a lubricant, a binder, an emulsifier, a thickener, a humectant, a stabilizer, a preservative, a solvent, a solubilizing agent, a suspending agent, a buffer, a pH adjuster, a gelling agent, an adhesive, an antioxidant, an absorption enhancer, an irritation suppressant, an antiseptic, a chelator and a dispersant.

The amount of the surfactant compounded in the formulation of the present invention can be appropriately set as long as the effect of the present invention is exerted. For example, the weight ratio between the surfactant and the active ingredient compounded in the formulation of the present invention can be 1:3 to 1:100. Such a configuration can allow the formulation to be not only excellent in morphological stability, but also excellent in absorbability. In this regard, the weight ratio between the surfactant and the active ingredient compounded in the formulation of the present invention is preferably 1:5 to 1:70.

### 2. Method for Producing Formulation

The formulation of the present invention can be, but are not particularly limited, for example, produced as follows.

First, the particle of the present invention can be, for example, produced by a method including the following step (1), without any particular limitation thereto.

(1) a step of homogenizing a water (W) phase containing an active ingredient and an oil (O) phase containing a surfactant to provide a W/O emulsion

In step (1), the pH of the water phase is preferably reduced because morphological stability of the resulting particle is excellent. Conventionally, homogenization has been often performed near neutral pH for the purpose of dissolution of the stabilizer, and the like. The reduction in pH is preferably 1 or more, more preferably 2 or more, further preferably 3 or more, still more preferably 4 or more based on that of a water phase containing no pH adjuster. The above pH adjuster can be, if necessary, used for this pH adjustment.

The method for producing a formulation of the present invention may further contain the following step, if necessary.

(2) a step of drying the W/O emulsion obtained in step (1).

The method for drying the W/O emulsion containing an active ingredient in the water phase in step (2) is not particularly limited as long as a solvent (aqueous solvent and oil solvent) in the emulsion can be removed, and examples include lyophilizing and drying under reduced pressure, preferably lyophilizing.

The method for producing a formulation of the present invention may further contain the following step, if necessary.

(3) a step of dispersing the lyophilize product obtained in step (2) in a base.

Specific examples of the method for producing a formulation of the present invention include the following.

An active ingredient, and optionally, additive ingredient(s) such as a stabilizer, an absorption enhancer and an irritation suppressant are dissolved in a solvent such as pure water or phosphate buffer. The pH of the solvent is preferably adjusted by adding a pH adjuster, if necessary.

A solution of a surfactant, and optionally, additive ingredient(s) such as an irritation suppressant, an analgesic, an absorption enhancer and a stabilizer in a solvent such as cyclohexane, hexane or toluene is added to the solution obtained above, and the resultant is stirred by a homogenizer or the like to provide a W/O emulsion.

Thereafter, the W/O emulsion can be lyophilized or the like, and, if necessary, dispersed in a base such as isopropyl myristate and subjected to a filtration treatment with a filter or the like, chromatography, centrifugation, or the like, thereby preparing the particle of the present invention. Production of the particle can be confirmed using particle size measurement or an optical microscope.

The particle can be used to produce, for example, the following formulation. The particle can be added to and mixed with a base such as a liquid base or a base such as an ointment, and additive ingredient(s) such as an absorption enhancer, a thickener and a gelling agent at a predetermined ratio, thereby providing a composition containing the particle. The composition thus obtained can be used as it is, or can be used, depending on the intended use, in the form of being retained by lamination or immersion on a natural woven member such as gauze or absorbent cotton, a synthetic fiber woven member such as polyester or polyethylene, or a woven fabric or non-woven fabric processed by appropriately combining such members, or a permeable membrane or the like, and the resultant can be used by further covered with an adhesion covering material or the like.

The particle can also be used to produce an ophthalmic formulation. A pharmaceutically acceptable additive can be, if necessary, added to the ophthalmic solution by use of a general-purpose technique. The concentration of the active ingredient in the ophthalmic solution can be usually 0.0001 to 1% by weight, and is preferably 0.0005 to 0.5% by weight, particularly preferably 0.001 to 0.1% by weight. Such a formulation solution can be subjected to filtration sterilization or other sterilization. The sterilization method is not particularly limited as long as the resulting formulation solution can be sterilized, and is preferably filtration sterilization using a sterilization filter having a pore size of 0.1 to 0.5 μm.

Examples of other method for producing a formulation include solution coating. For example, first, the particle and the base in the present invention, as well as additive ingredient(s) such as an absorption enhancer, a thickener and a gelling agent are added to a solvent such as hexane, toluene or ethyl acetate at a predetermined ratio, and stirred to prepare a uniform solution. The solid concentration in the solution is preferably 10 to 80% by weight, more preferably 20 to 60% by weight. Next, a release liner (for example, a polyester film treated with silicone) can be uniformly coated with the solution containing the respective ingredients by use of a coater such as a knife coater, a comma coater or a reverse coater and dried to complete an active ingredient-containing layer, and a support can be laminated on the layer to thereby prepare the formulation. A release liner may be laminated on the surface of the layer after formation of the layer on a support, depending on the type of the support.

The formulation thus obtained is appropriately cut into an elliptical, circular, square or rectangular shape, depending on the intended use. An adhesive layer or the like may also be, if necessary, provided on a surrounding portion.

3. Application of formulation

The formulation of the present invention can be used in wide applications including an external medicine and a cosmetic product, depending on the type of the active ingredient. The formulation of the present invention, without any particular limitation, can be used as a percutaneous absorption formulation. In this case, the formulation is usually used so as to be persistent for one day to one week, and in a preferable aspect, so as to be applied once per day to week.

When the formulation of the present invention is an external medicine, a disease of interest varies depending on the type of the active ingredient.

The percutaneous absorption formulation of the present invention can be used in a tape (reservoir type, matrix type, and the like), an ointment, a lotion, an aerosol, a plaster, an aqueous poultice, a cream, a gel, an aerosol, a patch, a microneedle, and the like, without any particular limitation.

Hereinafter, the present invention will be described in detail with reference to Examples and Test Examples, but the present invention is not intended to be limited to such Examples.

### EXAMPLES

### Production Example 1

In 40 g of pure water was dissolved 200 mg of donepezil hydrochloride (pKa=8.9), and a solution of 3.0 g of sucrose erucic acid ester (produced by Mitsubishi-Chemical Foods Corporation, ER-290; HLB value: 2) in 80 g of cyclohexane was added thereto. Four of such solutions were prepared, one thereof was not subjected to pH adjustment (pH: 5.47), and the remaining three solutions were subjected to pH adjustment at room temperature so that low pH (pH: 1.2 and pH: 3.9) and high pH (pH: 6.94) were achieved, respectively. The pH adjusters used were an aqueous 1 N hydrochloric acid solution and an aqueous 1 N sodium hydroxide solution. The respective amounts of the pH adjusters used in adjustment of the low pH solutions and the high pH solution were 5.4 mL, 1.3 mL and 1.3 mL.

Each of these four solutions was stirred by a homogenizer (10000 rpm) at room temperature. Thereafter, each of the solutions was lyophilized for two days to thereby prepare the particle of the present invention. In 925 mg of isopropyl myristate (produced by Wako Pure Chemical Industries, Ltd.) was dispersed 75 mg of the resulting particle, to thereby produce the formulation of the present invention.

### Production Example 2

In 40 g of pure water was dissolved 200 mg of vardenafil hydrochloride (pKa=8.8), and a solution of 3.0 g of sucrose erucic acid ester (produced by Mitsubishi-Chemical Foods Corporation, ER-290; HLB value: 2) in 80 g of cyclohexane was added thereto. Three of such solutions were prepared, one thereof was not subjected to pH adjustment (pH: 2.74), and the remaining two solutions were subjected to pH adjustment at room temperature so that low pH (pH: 1.2) and high pH (pH: 5.53) were achieved, respectively. The pH adjusters used were an aqueous 1 N hydrochloric acid solution and an aqueous 1 N sodium hydroxide solution. The respective amounts of the pH adjusters used in adjustment of the low pH solution and the high pH solution were 1.2 mLg and 4.1 mLg.

Each of these three solutions was stirred by a homogenizer (10000 rpm) at room temperature. Thereafter, each of the solutions was lyophilized for two days to thereby prepare the particle of the present invention. In 925 mg of isopropyl myristate (produced by Wako Pure Chemical Industries, Ltd.) was dispersed 75 mg of the resulting particle, to thereby produce the formulation of the present invention.

### Production Example 3

In 40 g of pure water was dissolved 200 mg of rivastigmine tartrate (pKa=8.9), and a solution of 3.0 g of sucrose erucic acid ester (produced by Mitsubishi-Chemical Foods Corporation, ER-290; HLB value 2) in 80 g of cyclohexane was added thereto. Four of such solutions were prepared, one thereof was not subjected to pH adjustment (pH: 3.53), and the remaining three solutions were subjected to pH adjustment at room temperature so that low pH (pH: 1 and pH: 2) and high pH (pH: 10) were achieved, respectively. The pH adjusters used were an aqueous 1 N hydrochloric acid solution and an aqueous 1 N sodium hydroxide solution. The respective amounts of the pH adjusters used in adjustment of the low pH solutions and the high pH solution were 3.8 mL, 1.2 mL and 6.8 mL.

Each of these four solutions was stirred by a homogenizer (10000 rpm) at room temperature. Thereafter, each of the solutions was lyophilized to thereby prepare the particle of the present invention. In 925 mg of isotridecyl isononanoate (produced by Kokyu Alcohol Kogyo Co., Ltd., KAK139) was dispersed 25 mg of the resulting particle, to thereby produce the formulation of the present invention.

### Production Example 4

In 40 g of pure water was dissolved 200 mg of duloxetine hydrochloride (pKa=9.7). Four of such solutions were prepared, one thereof was not subjected to pH adjustment (pH: 3.25), and the remaining three solutions were subjected to pH adjustment at room temperature so that low pH (pH: 1, and pH: 2) and high pH (pH: 7.47) were achieved, respectively. The pH adjusters used were an aqueous 1 N hydrochloric acid solution and an aqueous 1 N sodium hydroxide solution. The respective amounts of the pH adjusters used in adjustment of the low pH solutions and the high pH solution were 3.8 mL, 1.2 mL and 5.4 mL.

A solution of 3.0 g of sucrose erucic acid ester (produced by Mitsubishi-Chemical Foods Corporation, ER-290; HLB value: 2) in 80 g of cyclohexane was added to each of these four solutions, and the resultant was stirred by a homogenizer (10000 rpm) at room temperature. Thereafter, each of the solutions was lyophilized for two days to thereby prepare the particle of the present invention. In 925 mg of isotridecyl isononanoate (produced by Kokyu Alcohol Kogyo Co., Ltd., KAKI39) was dispersed 75 mg of the resulting particle, to thereby produce a formulation.

Morphological Stability Test

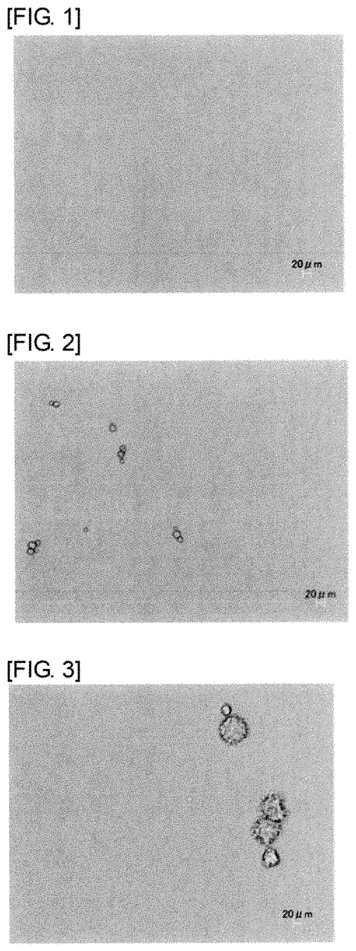

Each of the formulations obtained in Production Examples 1 to 4 was subjected to a morphological stability test. Specifically, each of the formulations was stored at 60° C., and evaluation was performed as to how many days after the start of storage a morphological change relative to the initial shape was observed. Such observation was performed by an optical microscope. The results are shown in Table 1 and FIGS. 1 to 3. In Table 1, a case where the stability was improved as compared with a case of no pH adjustment is represented as Example, and on the other hand, a case of no improvement is represented as Comparative Example together with a case of no pH adjustment. FIGS. 1 to 3 sequentially illustrate images of the formulations in Example 1, Comparative Example 1 and Comparative Example 2, after 2 days at 60° C., observed by an optical microscope, respectively. While no morphological change was caused in Example 1, a morphological change was caused in each of Comparative Examples 1 and 2.

TABLE 1

| | | | | | | Number of days | Difference |
|---|---|---|---|---|---|---|---|
| | | | | pH of | Difference in | X taken for | in number |
| | | Example/ | | agent | pH based | morphological | of days X |
| Production | Active | Comparative | | solution in | on pH | change to be | based on pH |
| Example | ingredient | Example | pH Adjuster | production | not adjusted | observed | not adjusted |
| Production | Donepezil | Example 1 | Aqueous 1N | 1.2 | −4.27 | 3 Days | +1 |
| Example 1 | hydrochloride | | hydrochloric | | | | |
| | | | acid solution | | | | |
| | | Example 2 | Aqueous 1N | 3.9 | −1.57 | 3 Days | +1 |
| | | | hydrochloric | | | | |
| | | | acid solution | | | | |
| | | Comparative Example 1 | None | 5.47 | 0 | 2 Days | 0 |
| | | Comparative Example 2 | Aqueous 1N sodium | 6.94 | +1.47 | 1 Day | −1 |
| | | | hydroxide solution | | | | |
| Production | Vardenafil | Example 3 | Aqueous 1N | 1.2 | −1.54 | 3 Days | +1 |
| Example 2 | hydrochloride | | hydrochloric | | | | |
| | | | acid solution | | | | |
| | | Comparative Example 3 | None | 2.74 | 0 | 2 Days | 0 |
| | | Comparative Example 4 | Aqueous 1N sodium | 5.53 | +2.79 | 1 Day | −1 |
| | | | hydroxide solution | | | | |
| Production | Rivastigmine | Example 4 | Aqueous 1N | 1 | −2.53 | 7 Days | +2 |
| Example 3 | tartrate | | hydrochloric | | | | |
| | | | acid solution | | | | |
| | | Example 5 | Aqueous 1N | 2.0 | −1.53 | 6 Days | +1 |
| | | | hydrochloric | | | | |
| | | | acid solution | | | | |
| | | Comparative Example 5 | None | 3.53 | 0 | 5 Days | 0 |
| | | Comparative Example 6 | Aqueous 1N sodium | 10 | +6.47 | 1 Day | −4 |
| | | | hydroxide solution | | | | |
| Production | Duloxetine | Example 6 | Aqueous 1N | 1 | −2.25 | 7 Days | +2 |
| Example 4 | hydrochloride | | hydrochloric | | | | |
| | | | acid solution | | | | |
| | | Example 7 | Aqueous 1N | 2.7 | −1.55 | 7 Days | +2 |
| | | | hydrochloric | | | | |
| | | | acid solution | | | | |
| | | Comparative Example 7 | None | 3.25 | 0 | 5 Days | 0 |
| | | Comparative Example 8 | Aqueous 1N sodium | 7.47 | +4.22 | 2 Days | −3 |
| | | | hydroxide solution | | | | |

1. A particle comprising an active ingredient, a surfactant and a pH adjuster.

2. The particle according to claim 1, wherein
the particle comprises a first fraction and a second fraction, and
the first fraction comprises the active ingredient and the pH adjuster.

3. The particle according to claim 1, wherein the pH adjuster is at least one pH adjuster selected from the group consisting of an inorganic acid and an organic acid, and a salt thereof.

4. The particle according to claim 1, wherein the pH adjuster is at least one pH adjuster selected from the group consisting of hydrochloric acid and a hydrochloride.

5. The particle according to claim 1, comprising 0.01 to 2% by weight of the pH adjuster based on the entire particle.

6. The particle according to claim 1, wherein the active ingredient is hydrophilic.

7. A formulation comprising the particle according to claim 1.

8. A method for producing a particle comprising an active ingredient, a surfactant and a pH adjuster, the method comprising:
(1) a step of preparing a water (W) phase comprising an active ingredient and a pH adjuster;
(2) a step of homogenizing the water (W) phase and an oil (O) phase comprising a surfactant to provide a W/O emulsion; and
(3) a step of drying the W/O emulsion.
